(19) United States
(12) Patent Application Publication
(10) Pub. No.: US 2020/0155175 A1
(43) Pub. Date: May 21, 2020

(54) TRACKED SURGICAL TOOL WITH CONTROLLED EXTENSION

(71) Applicant: Smith & Nephew, Inc., Memphis, TN (US)

(72) Inventors: Constantinos NIKOU, Monroeville, PA (US); Branislav JARAMAZ, Pittsburgh, PA (US); David Michael DAVIDSON, Pittsburgh, PA (US)

(21) Appl. No.: 16/751,911

(22) Filed: Jan. 24, 2020

Related U.S. Application Data
(63) Continuation of application No. 16/478,314, filed on Jul. 16, 2019.
(60) Provisional application No. 62/446,651, filed on Jan. 16, 2017.

(51) Int. Cl. A61B 17/17; A61B 17/16; A61B 90/00; A61B 34/10

(52) U.S. Cl. CPC A61B 17/1703; A61B 17/1764; A61B 17/1675; A61B 2017/00212; A61B 17/1626; A61B 90/06; A61B 34/10; A61B 17/1633

(57) ABSTRACT

A system, device, and method for controlling operation of a surgical tool (214) during a surgical procedure are described. For example, the system includes a tool assembly (210) and a surgical system (100). The tool assembly includes a rotating tool (214) and a sleeve (212) for holding the rotating tool. The surgical system includes a navigation system (200) configured to track at least a portion of the rotating tool and determine position information for the rotating tool, an alignment module (222) configured to receive the position information and determine whether the rotating tool is in a proper position for drilling a hole into a target bone (240), a robotic control component (220) configured to actuate and advance the rotating tool if the rotating tool is in the proper position, and a monitor (226) configured to determine if the rotating tool is advancing into the target bone along a predetermined path.

### CLAIM OF PRIORITY

This application claims the benefit of priority to U.S. Provisional Application No. 62/446,651, titled “Tracked Surgical Tool with Controlled Extension,” filed Jan. 16, 2017, which is incorporated herein by reference in its entirety.

### TECHNICAL FIELD

The present disclosure relates generally to apparatus, methods, and systems for computer-aided orthopedic surgery. More specifically, the present disclosure relates to a controlled system for actuating tools to be used in computer-aided surgical procedures.

### BACKGROUND

The use of computers, robotics, and imaging to aid orthopedic surgery is known in the art. There has been a great deal of study and development of computer-aided navigation and robotic systems used to guide surgical procedures. For example, a precision freehand sculptor (PFS) can employ a robotic surgery system to assist the surgeon in accurately cutting a bone into a desired shape. In procedures such as total knee replacement, computer-aided surgery techniques have been used to improve the accuracy and reliability of the surgery. Orthopedic surgery guided by images has also been found useful in preplanning and guiding the correct anatomical position of displaced bone fragments in fractures, allowing a good fixation by osteosynthesis.

Cut guides or cutting blocks can be used in an orthopedic surgical procedure to assist a surgeon in cutting or modifying some portions of a target bone. For example, in joint replacement surgeries such as total hip replacement (THR) or total knee replacement (TKR), the preparation of the bones can involve temporarily affixing saw guide cutting blocks to the bones so that a reciprocating saw blade can be held steady along its intended path. Placement of these blocks can be guided by manual instrumentation or through the use of jigs.

The proper alignment of manual instruments, including guides and jigs, is cumbersome and, further, is limited to information obtainable through mechanical and visual referencing means. Manual instruments are also expensive to purchase and manage, and there are significant operational costs in maintaining and cleaning such instruments. Some manual instruments, such as mechanical referencing instruments, can add to the burden of managing the guides and jigs.

Existing surgical navigation systems typically use optical trackers to align guides, jigs, or couplers that interface with the jigs. The use of such systems can provide surgeons with more information than traditional mechanical jigs. The information can include range of motion capture, intraoperatively defined anatomical landmarks, and preoperatively defined anatomic landmarks. Existing systems can align guides precisely, but interfacing trackers or couplers to guides or jigs for alignment purposes creates an extra unnecessary step.

Pilot holes can be created to help align guides, but the drilling of pilot holes with a handheld drill is challenging because surgeons may not be able to hold the drill at the proper angle and/or position before starting the drill. Additionally, it can difficult to advance a drill bit along the proper trajectory without having the drill bit move out of alignment with the preferred trajectory. Moreover, it is difficult to start a drill by hand without skipping or moving along a bone surface.

Known surgical techniques can involve the use of a plunge stabilizer to hold a drill steady. However, it is difficult to hold the proper alignment while plunging the drill into contact with the bone. For these reasons, an improved surgical drilling system is needed.

### SUMMARY

There is provided a system for controlling operation of a surgical tool during a surgical procedure. The system includes a tool assembly and a surgical system. The tool assembly includes a rotating tool and a sleeve for holding the rotating tool, the sleeve including at least one anchoring feature configured to anchor the tool assembly to a target bone prior to actuation of the rotating tool. The surgical system includes a navigation system configured to track at least a portion of the rotating tool and determine position information for the rotating tool, an alignment module configured to receive the position information and determine whether the rotating tool is in a proper position for drilling a hole into a target bone, a robotic control component configured to actuate and advance the rotating tool if the alignment module determines the rotating tool is in the proper position, and a monitor configured to determine if the rotating tool is advancing into the target bone along a predetermined path.

In some embodiments, the surgical system is configured to determine a surgical plan for the surgical procedure, determine at least one hole to be drilled into the target bone according to the surgical plan, and determine the predetermined path for the at least one hole to be drilled based upon the surgical plan. In some additional embodiments, the surgical plan defines a knee replacement procedure.

In some embodiments, the system for includes a trigger configured to receive an input from a user and facilitate actuation of the rotating tool by the robotic control component. In some additional embodiments, the trigger includes at least one of a foot pedal operably connected to the surgical system, a vocal trigger operably connected to the surgical system, and a pushbutton integrated into the tool assembly.

In some embodiments, the monitor is further configured to monitor alignment of the rotating tool during drilling of the hole and stop the rotating tool if the rotating tool is out of alignment.

In some embodiments, the monitor is further configured to determine a depth of the rotating tool during drilling of the hole and stop the rotating tool when the rotating tool reaches a predetermined depth.

In some embodiments, the rotating tool includes a tool tip for cutting the target bone during drilling of the hole. In some additional embodiments, the tool tip includes at least one of a drill bit and a cutting bur.

In some embodiments, the sleeve comprises an elongated bore through which the rotating tool can extend from and be retracted into.

In some embodiments, the sleeve is configured to position the rotating tool away from the target bone prior to actuation of the rotating tool.

There is also provided a device for controlling operation of a surgical tool during a surgical procedure. The device includes a processing device operably connected to a computer readable medium configured to store one or more instructions. When executed, the instructions cause the processing device to track at least a portion of a rotating tool during the surgical procedure, determine position information for the rotating tool, determine, based upon the position information, whether the rotating tool is in a proper position for drilling a hole into a target bone such that at least a portion of the rotating tool is anchored to the target bone prior to actuation of the rotating tool, actuate and advance the rotating tool if the rotating tool is in the proper position, and determine if the rotating tool is advancing into the target bone along a predetermined path.

In some embodiments, the one or more instructions comprise additional instructions that, when executed, cause the processing device to determine a surgical plan for the surgical procedure, determine at least one hole to be drilled into the target bone according to the surgical plan, and determine the predetermined path for the at least one hole to be drilled based upon the surgical plan. In some additional embodiments, the surgical plan defines a knee replacement procedure.

In some embodiments, the one or more instructions comprise additional instructions that, when executed, cause the processing device to monitor alignment of the rotating tool during drilling of the hole and stop the rotating tool if the rotating tool is out of alignment.

In some embodiments, the one or more instructions comprise additional instructions that, when executed, cause the processing device to determine a depth of the rotating tool during drilling of the hole and stop the rotating tool when the rotating tool reaches a predetermined depth.

There is also provided a method for controlling operation of a surgical tool during a surgical procedure. The method includes tracking, by a navigation system operably connected to a surgical system, at least a portion of a rotating tool during the surgical procedure; determining, by the navigation system, position information for the rotating tool; receiving, by an alignment module operably connected to the navigation system, the position information; determining, by the alignment module, whether the rotating tool is in a proper position for drilling a hole into a target bone based upon the position information such that at least a portion of the rotating tool is anchored to the target bone prior to actuation of the rotating tool; actuating and advancing, by a robotic control component operably connected to the alignment module, the rotating tool if the rotating tool is in the proper position; and determining, by a monitor operably connected to the robotic control component, if the rotating tool is advancing into the target bone along a predetermined path.

In some embodiments, the method further includes determining a surgical plan for the surgical procedure, determining at least one hole to be drilled into the target bone according to the surgical plan, and determining the predetermined path for the at least one hole to be drilled based upon the surgical plan. In some additional embodiments, the surgical plan defines a knee replacement procedure.

In some embodiments, the method further includes monitoring alignment of the rotating tool during drilling of the hole and stopping the rotating tool if the rotating tool is out of alignment.

In some embodiments, the method further includes determining a depth of the rotating tool during drilling of the hole and stopping the rotating tool when the rotating tool reaches a predetermined depth.

The example embodiments as described above can provide various advantages over prior techniques. For example, the techniques as taught herein can reduce the time spent mounting a cut guide onto a target bone. The techniques also provide for more accurately drilling properly located and sized holes for pinning cut guides in the optimal position for a specific implant component.

Further features and advantages of at least some of the embodiments of the present disclosure, as well as the structure and operation of various embodiments of the present disclosure, are described in detail below with reference to the accompanying drawings.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 is a block diagram depicting a system for providing surgical navigation to ensure an orthopedic procedure is consistent with a surgical plan.

FIG. 2 is an illustration of an operating room with a system employing a navigated robotic surgical tool in accordance with certain embodiments of the invention.

FIG. 3 is a block diagram depicting a system implemented as one embodiment of the invention.

FIG. 4 is a cross sectional view in side elevation illustrating a portion of the system generally described in FIG. 3.

FIG. 5 is a cross sectional view in side elevation illustrating a portion of the system generally described in FIG. 3.

FIG. 6 is a block diagram depicting a system implemented as another embodiment of the invention.

FIG. 7 illustrates a perspective view of another embodiment of the invention.

FIG. 8 illustrates a process in accordance with an embodiment of the invention.

FIG. 9 illustrates an alternative process in accordance with certain embodiments of the invention.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

This disclosure describes certain embodiments of navigated surgical systems and processes for determining when a tool is properly positioned and aligned on a work surface to allow for user-prompted initiation of the tool, such as for gradual extension of a drill bit along its axis. In the following description, for purposes of explanation, numerous specific details are set forth in order to provide a thorough understanding of example embodiments. It will be evident to one skilled in the art, however, that embodiments can be practiced without these specific details.

For the purposes of this specification, the term “implant” is used to refer to a prosthetic device or structure manufactured to replace or enhance a biological structure, either permanently or on a trial basis. For example, in a knee replacement procedure, an implant can be placed on one or both of the tibia and femur. While the term “implant” is generally considered to denote a man-made structure (as contrasted with a transplant), for the purposes of this specification, an implant can include a biological tissue or material transplanted to replace or enhance a biological structure.

The invention can be implemented in systems that are particularly adapted for implant surgery and, in particular, knee implant surgery. The inside of a femoral knee implant, for example, typically includes three flat surfaces. The end of the patient's femur can be prepared to receive the implant by cutting three planar surfaces at different angles that match the inside of the femoral implant. Typically, these cuts are made with a sagittal saw. Preparation of the bone traditionally involves affixing cut guides to the bone to ensure each cut is made along the intended plane. The placement of these cut guides can be guided by manual instrumentation or by a surgical navigation system.

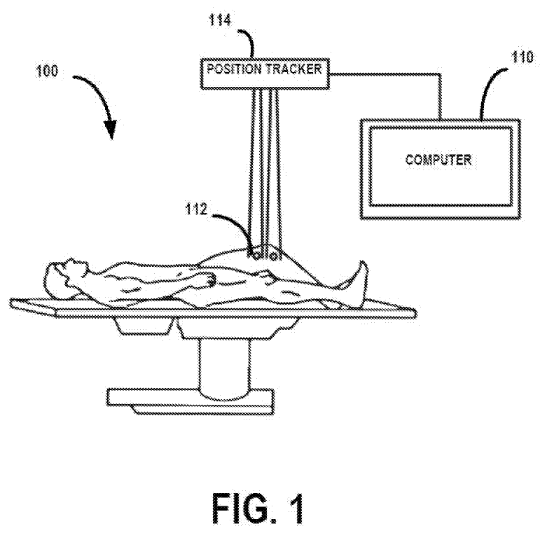

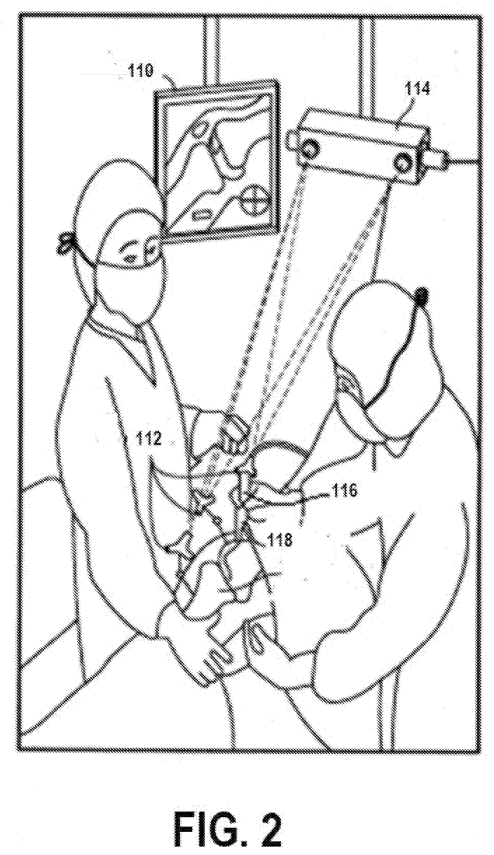

FIGS. 1 and 2 illustrate components of a surgical navigation system 100 for controlling cutting elements during a procedure to prepare one or more operative bones for placement of an implant, according to certain embodiments. The surgical navigation system 100 can assist a surgeon in cutting or modifying some portions of a target bone. For example, in joint replacement surgeries such as total or partial knee replacement surgeries, the surgical navigation system 100 can be used to selectively and optimally cut portions of the ends of the target bones and replace those portions with endoprosthetic implants.

FIG. 1 illustrates components of a surgical navigation system 100 that can be configured to perform robotically-assisted surgical procedures. The surgical navigation system 100 can assist a surgeon in performing certain surgical procedures, such as knee replacement revision surgery, but can also be used for procedures involving other joints including, for example, hip replacement surgeries.

In certain embodiments, the surgical navigation system 100 can include a computer system 110 to provide a display for viewing location data provided by optical trackers 112 as read by a position tracker 114. The optical trackers 112 and position tracker 114 can provide data relevant to the precise location of the bones in the knee joint once their location is registered with the system 100. In certain embodiments, the position tracker 114 can detect tracking spheres located on the optical trackers 112 in order to gather location data for a patient. In some implementations, the optical trackers 112 can be arranged into an array having fixed dimensions known by the position tracker 114 and/or the surgical navigation system 100. By monitoring the movement of the optical trackers 112 in the workspace, as well as the relative distance between the individual optical trackers (e.g. the individual tracking spheres), the surgical navigation system 100 can monitor and track the position and movement of the optical trackers throughout the workspace. From this, the surgical navigation system 100 can determine, for example, a position of an object such as a tool or a patient's bone that the optical trackers 112 are attached to.

For example, the optical trackers 112 can be mounted such that information related to femur and tibia positions can be collected during a knee replacement procedure. The position tracker 114 can be any suitable tracker, such as active trackers, passive trackers, optical trackers, electromagnetic trackers, infrared camera systems, or other similar systems.

FIG. 2 illustrates an exemplary optical surgical navigation setup. Position tracker 114 and optical trackers 112 can be used to perform surgical navigation as described above. The optical trackers 112 can be rigidly attached to any object (such as the surgical tool 116 or operative bones) that the surgeon wishes to track during the procedure. The position tracker 114 can continuously monitor the workspace during the procedure. The optical trackers 112 can be detected and tracked in workspace images collected by the position tracker 114. Using a known rigid spatial relationship of the optical trackers 112 to a surgical tool 116, the position of a surgical tool tip 118 in a three-dimensional space can be tracked by the position tracker 114 and continuously provided to the computer system 110. If the operative bones are also being tracked, the computer system 110 can continuously display the surgical tool 116 and/or the tip 118 location relative to the patient's anatomy. In certain implementations, the surgical tool 116 can be actuated when in an appropriate position according to a previously determined surgical plan.

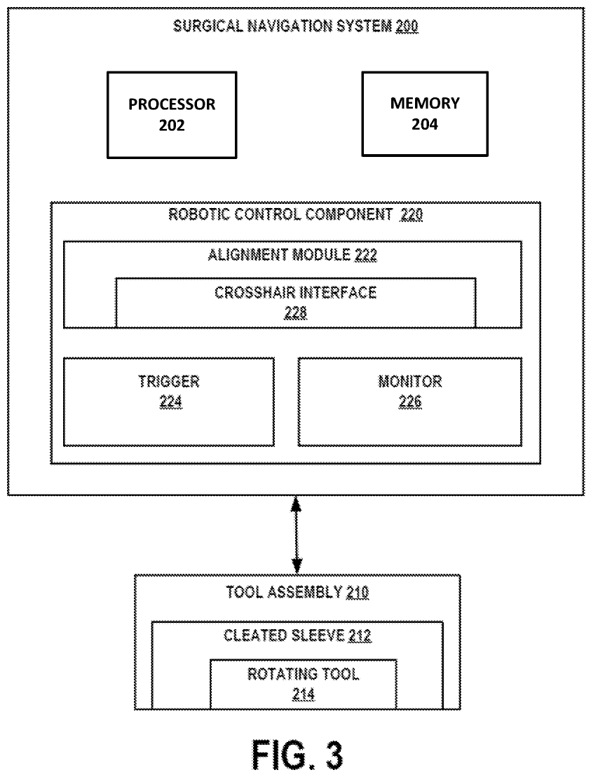

FIG. 3 depicts a surgical navigation system, generally designated as 200, consistent with certain embodiments as described herein. The surgical navigation system 200 can be operably connected to a surgical tool assembly 210 having a cleated sleeve 212 and a rotating tool 214. In some implementations, the surgical navigation system 200 can be operably connected to the tool assembly via one or more wires. In some implementations, the surgical navigation system 200 can include a tracking component similar to position tracker 114 as described above. In such an example, the tool assembly 210 can include a set of optical trackers configured to be tracked by the surgical navigation system 200, thereby providing the surgical navigation system with location information related to the current location of the tool assembly (and, by extension, the rotating tool 214). The surgical navigation system 200 can determine position and orientation information for the tool assembly 210 based upon analysis of the location information. Thus, the surgical navigation system 200 can determine the position and orientation (and resulting alignment) of the rotating tool 214 prior to operation of the rotating tool.

In some examples, the surgical navigation system 200 can include a robotic control component 220 configured to control operation of the surgical tool assembly 210. It is to be appreciated that embodiments of the surgical navigation system 200 and/or the robotic control component 220 can be implemented by various types of operating environments, computer networks, platforms, frameworks, computer architectures, and/or computing devices.

The surgical navigation system 200 and/or the robotic control component 220 can include one or more processors and memory devices, as well as various input devices, output devices, communication interfaces, and/or other types of devices. The surgical navigation system 200 and/or the robotic control component 220 can include a combination of hardware and software.

The surgical navigation system 200 and/or the robotic control component 220 can implement and utilize one or more program modules. Additionally or alternatively, the surgical navigation system 200 and/or the robotic control component 220 can implement and utilize one or more program modules or similar sets of instructions contained in a computer readable medium or memory (e.g., memory 204) for causing a processing device (e.g., processor 202) to perform one or more operations. Generally, program modules include routines, programs, objects, components, data structures, etc., that perform particular tasks or implement particular abstract data types.

The surgical navigation system 200 and/or the robotic control component 220 can be implemented by one or more computing devices, including computers, PCs, server computers configured to provide various types of services and/or data stores in accordance with aspects of the described subject matter. Exemplary server computers can include, without limitation: web servers, front end servers, application servers, database servers, domain controllers, domain name servers, directory servers, and/or other suitable computers. Components of surgical navigation system 200 and/or the robotic control component 220 can be implemented by software, hardware, firmware or a combination thereof.

The surgical navigation system 200 can be implemented with a robotic-assisted bone preparation system, such as the total knee arthroplasty application that is implemented through the NAVIO® system. NAVIO is a registered trademark of BLUE BELT TECHNOLOGIES, INC. of Pittsburgh, Pa. In certain embodiments, the surgical navigation system 200 can include other tracking systems, components, or modules.

The tool assembly 210 can include a cleated sleeve 212 that operates in conjunction with a rotating tool 214. In some implementations, the tool assembly 210 can be navigated and/or tracked through interaction with the surgical navigation system 200.

In some examples, the surgical navigation system can include a trigger 224 configured to provide a manual control for initiating and stopping operation of the tool assembly 210. In certain embodiments, the rotating tool 214 is a drill and the trigger 224 is a foot pedal. In some examples, the trigger 224 can be implemented as a vocal trigger configured to monitor for a voice command or another similar audible command for triggering operation of the rotating tool 214.

It should be noted that the trigger 224 is shown as a component of the surgical navigation system 200 by way of example only. In some implementations, the trigger 224 can be integrated into the tool assembly 210. For example, the trigger 224 can be a pushbutton positioned on the base of the rotating tool 214, such as a cutting bur, such that the tool assembly 210 can be hand-operated by the surgeon.

In certain embodiments, the robotic control component 220 can include an alignment module 222 and a monitor 226. The alignment module 222 can be configured to implement a crosshair interface 228. The crosshair interface 228 can be configured to communicate with the monitor 226 to provide current location information for the rotating tool 214 during a surgical procedure (determined from, for example, the position and orientation information determined by the surgical navigation system 200 as described above). The monitor 226 can analyze the location information to determine if the rotating tool 214 is correctly positioned and/or aligned. The surgical navigation system 200 can provide a surgeon with information and/or instructions to align the rotating tool 214 with an intended operative location and trajectory before the rotating tool can be operated. For example, a display operably connected to the surgical navigation system 200 can display location and trajectory information to the physician as well as instructions or movement information for properly positioning and aligning the rotating tool 214.

In certain implementations, the robotic control component 220 can be configured to communicate with the tool assembly 210 to advance the rotating tool 214 to a position that is adjacent to a work surface. Known mechanisms for this advancement include lead screws, ball screws, linear actuators and worm gears. In certain embodiments, the cleated sleeve 212 can be configured and positioned to hold the rotating tool 214 at a predetermined distance in relation to the work surface without actually touching the surface. In other embodiments, the rotating tool 214 can extend beyond the cleated sleeve 212 to ensure a proper starting position prior to operation of the rotating tool.

In certain implementations, the surgical navigation system 200 can communicate tracking data to the monitor 226. The monitor 226 can determine when the tool assembly 210 is in the proper alignment and location based upon the tracking data. In certain embodiments, the rotating tool 214 can commence operation after it has been placed in the proper position for a predetermined length of time. In other embodiments, the surgical navigation system 200 can signal to the user that the rotating tool is in the correct position, but will not begin operation until the trigger 224 is actuated.

As discussed, in certain embodiments, the robotic control component 220 can activate the tool assembly 210 to automatically rotate the rotating tool 214 and advance the rotating tool 214 linearly. After the rotating tool 214 begins advancing, the alignment module 222 can monitor the alignment of the rotating tool and the monitor 228 can control the depth to which the rotating tool 214 penetrates a work surface within a workpiece (i.e., a drilling surface within a bone).

In other embodiments, the robotic control component 220 can configure the trigger 224 to initiate movement of the rotating tool 214. Upon actuation, the rotating tool 214 can extend to move into contact with the work surface. In some embodiments, the rotating tool 214 does not begin rotating until it makes contact with the work surface.

In certain embodiments, once the rotating tool 214 is moved into contact with the work surface, the robotic control component 220 can implement the crosshair interface 228 for aligning the rotating tool 214 so that a cut or hole made by the rotating tool is consistent with a predetermined surgical plan. In some implementations, the tool assembly 210 can further include adjustment means for making small adjustments to the position of the rotating tool 214. For example, the tool assembly 210 can include micro-servos or other small scale mechanical devices configured to adjust a position of the rotating tool 214 in response to an instruction from, for example, the robotic control component 220 and/or the alignment module 222.

In some examples, the robotic control component 220 can track the position of the crosshair interface 228 to determine whether the rotating tool 214 has remained properly positioned and aligned during cutting. Thus, through the use of the crosshair interface 228, the robotic control component 220 can overcome some of the limitations of conventional navigation systems when advancing the navigated or tracked rotating tool 214.

The robotic control component 220 configures and implements the monitor 226 to communicate with the crosshair interface 228 to ensure that the rotating tool 214 is properly aligned during a drilling operation or during another similar operation. Specifically, the monitor 226 can detect when the rotating tool 214 fails to maintain the proper alignment and can be configured to correct the alignment or to compensate for the misalignment. In some implementations, the monitor 226 can also be configured to stop the robotic tool 214 when it fails to maintain the proper alignment. Alternatively, the monitor 226 can be configured to retract the rotating tool 214 when it fails to maintain the proper alignment.

In an illustrative example, it is often necessary in joint replacement surgery to drill one or more holes in a patient's bone so that a cut guide can be attached. The drilling portions of the surgical plan can be determined by calculating a chain of coordinate transforms between an optical tracker mounted to the bone, to an implant cut shape for a chosen implant size and orientation in a surgical plan, to a set of cutting guide positions corresponding to the shape and position, and finally for the location and alignment of the peg holes needed to secure the cut guides in the proper calculated position that would make that shape. In this way, the surgical navigation system 200 can use the alignment module 222 to determine the location, depth, and alignment of any hole needed so that the cut guide can be attached to the bone in the correct place to enable the proper cuts to be made. In this manner, the surgical navigation system 200 provides for the easy alignment of cut guides that do not have to interface with manual instrumentation sets such as cut guide jigs. Such guides can be smaller or less intricate, which results in cost savings. Additionally, the invention provides a single system that can align multiple types of cut guides to support multiple surgeries and implant designs.

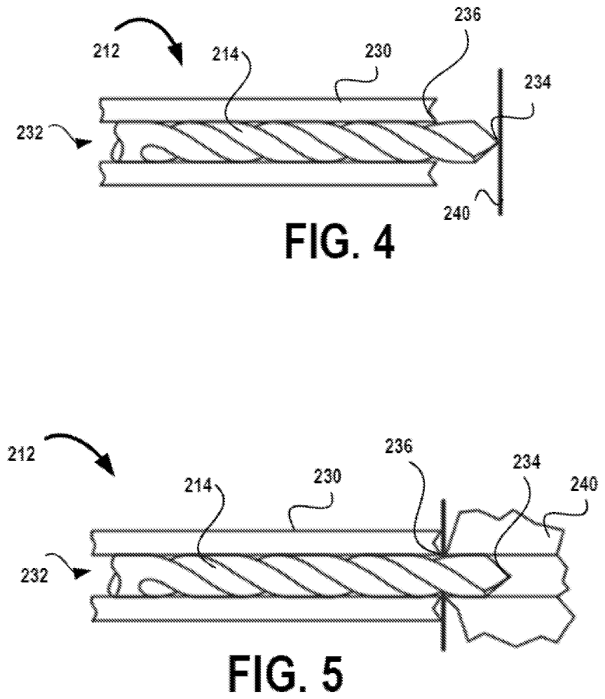

FIGS. 4 and 5 illustrate a cut-away illustration of the cleated sleeve 212 and rotating tool 214 in accordance with certain embodiments of the invention. The cleated sleeve 212 can include an essentially cylindrical tubular sleeve 230 forming an elongated bore 232 through which the rotating tool 214 can extend. In certain embodiments, a tip 234 can be biased in a slightly extended position beyond a mouth 236 of the bore 232 as illustrated in FIG. 4. Such an arrangement provides that the tip 234 can be pressed against the bone 240 while the rest of the tool assembly 210 is pivoted around that point until the alignment module 222 indicates the tool assembly 210 is aligned with the planned hole to be cut. In certain implementations, while keeping the tip 234 in contact with the bone 240, the rotating tool 214 can be retracted until the cleated sleeve 212 contacts the bone 240 to secure the position. The retraction could be programmed to be a quick retraction, to minimize error in the alignment. If the tool assembly 210 remains aligned, the rotating tool 214 can be actuated to create the appropriate hole as is demonstrated by FIG. 5.

In some examples, the cleated sleeve 212 can include a serrated edge configured to anchor the tool assembly 210 and/or the rotating tool 214 to the bone 240. In such examples, the cleated sleeve can provide an anchoring or engagement feature configured to stabilize and maintain the position of the tip 234 relative to the hole to be drilled into the bone 240, thereby reducing any potential movement of the rotating tool 214 or the tip when the tip contacts the bone. For example, the hole to be cut may be located on a sloped, curved, or otherwise angled portion of bone 240 where the tip 234 may be prone to sliding, skipping, or otherwise moving prior to engaging with the bone to cut the hole. By providing the cleated sleeve 212 with a serrated edge or another similar anchoring feature, the cleated sleeve can anchor the tool assembly 210 and/or the rotating tool 214 to the bone, thereby reducing or eliminating a chance of movement of the rotating tool when the tip 234 contacts the bone.

In an alternate embodiment, the tip 234 can be retracted into the bore 232 and the cleated sleeve 212 can be placed against the operative bone 240. The crosshair interface 228 within the alignment module 222 can be used to ensure the position and alignment of the tool assembly 210 is correct. Once actuated, such as via a trigger 224 or automatically actuated by, for example, the robotic control component 220, the rotating tool 214 can begin to spin. The robotic control component 220 can advance the rotating tool 214 until it hits the bone 240, thus reducing the chance that the tip 234 will be deflected from the intended location. The monitor 226 can guide the rotating tool 214 to ensure that the rotating tool 214 follows a predetermined trajectory. This allows a surgeon to focus on controlling the trajectory, while the robotic control component 220 controls the depth and speed of the rotating tool 214.

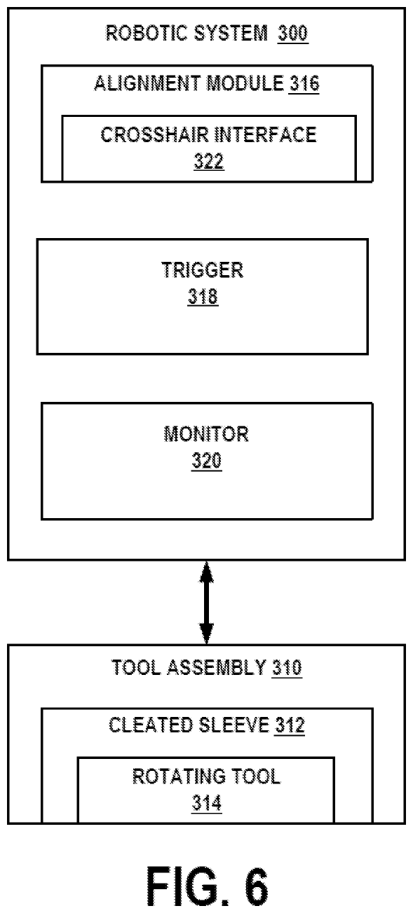

FIG. 6 illustrates a standalone robotic system 300 in accordance with another embodiment of the invention. In this exemplary embodiment, the standalone robotic system 300 can control a surgical tool assembly, generally designated by the numeral 310. Unlike the embodiment shown in FIGS. 3-5, the surgical tool assembly 310 is not connected to or otherwise controlled by a surgical navigation system, such as surgical navigation system 200.

The surgical tool assembly 310 can include a cleated sleeve 312 and a rotating tool 314. The cleated sleeve 312 can be the same as or otherwise equivalent to the cleated sleeve 212 that is shown in FIGS. 3-5. The rotating tool 314 can be any suitable rotating surgical tool, such as a drill, a bur, a screwdriver or other similar instrument. In some implementations, the surgical tool assembly 310 can be implemented with a tracking system (not shown).

The standalone robotic system 300 can include an alignment module 316, a trigger 318, and a monitor 320 that function in a manner that is similar to the alignment module 222, the trigger 224, and the monitor 226 as shown in FIGS. 3-5 and described above. The alignment module 316 can include a crosshair interface 322 that functions similarly to the crosshair interface 228 shown in FIGS. 3-5 and described above. The alignment module 316 and the monitor 320 can be configured to control the depth of any holes produced by the rotating tool 314.

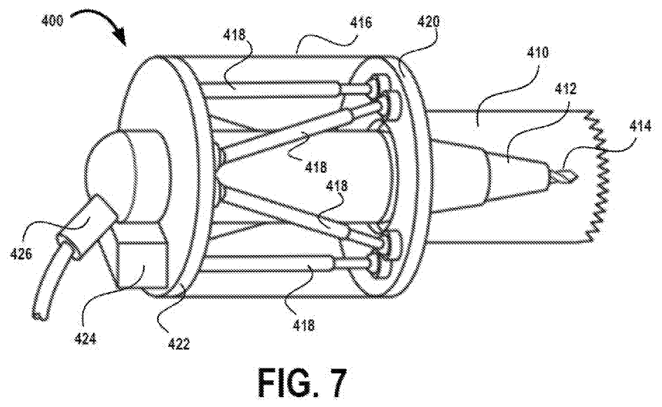

FIG. 7 illustrates a robotic tool assembly 400 in accordance with another embodiment of the invention. The robotic tool assembly 400 can be configured to be used within a surgical navigation system, such as surgical navigation system 200 shown in FIGS. 3-5, or as a standalone system with a robotic control system, such as robotic control system 300 and surgical tool assembly 310, shown in FIG. 6.

The robotic tool assembly 400 can include a cleated sheath 410 encircling a tool holder 412 and a rotating tool 414. Similar to the above description of the cleated sleeve 212, in certain implementations the cleated sheath can include a serrated edge (as shown in FIG. 7) or another similar engagement or anchoring feature for anchoring the robotic tool assembly 400 to a target bone prior to operation of the rotating tool 414.

In certain embodiments, the tool holder 412 can be a collet and the rotating tool 414 can be a drill. The rotating tool head 410 can be mounted on a platform 416 that includes a plurality of actuators 418 positioned between a proximal and a distal baseplate 422, 420 respectively. For example, the robotic tool assembly 400 can include six actuators 418. In certain implementations, the actuators 418 can be prismatic actuators, such as hydraulic jacks, or electric actuators, attached in pairs to three positions on the proximal baseplate 422. The actuators 418 can be positioned to cross over to three mounting points on the distal baseplate 420. It should be noted that six actuators is described by way of example only, and additional numbers of actuators can be used. Similarly, the positioning and mounting of the actuators 418 as shown in FIG. 7 and described herein is by way of example only.

The arrangement of the actuators 418 and the baseplates 420-422 can allow the tool head 410 and the rotating tool 414 to be moved with six degrees of freedom. The six degrees of freedom provide extra degrees of motion off-axis with the tool trajectory in order to possibly correct and/or compensate for the user deviating from a surgical plan.

The robotic tool assembly 400 can include a control box 424 and a power cord 426. The control box 424 can include a control unit for controlling the actuators, a computer device, and/or an interface to an external computer system that can control the robotic tool assembly 400. The power cord 426 can connect to an external power source. When placed in a proper location for a hole, certain embodiments of the robotic tool assembly 400 can be configured to automatically align the cutting tool 414 using the actuators 418. The robotic tool assembly 400 can further be configured or otherwise instructed to perform the cutting or drilling autonomously.

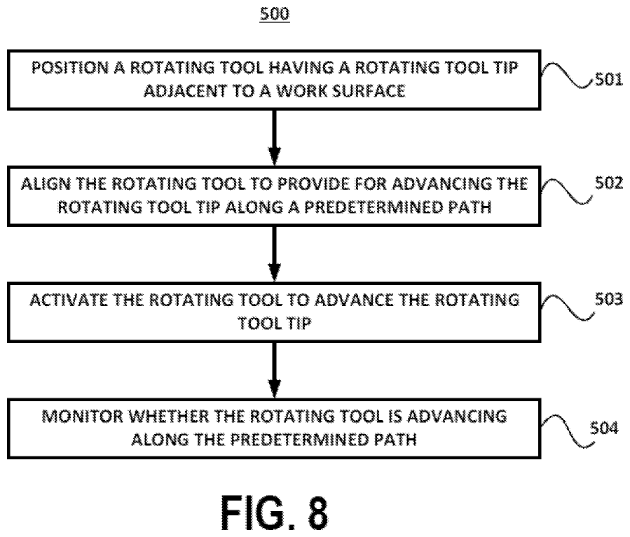

Referring to FIG. 8 with continuing reference to the foregoing figures, a process 500 is described as a sample robotically controlled drilling process in accordance with aspects of the subject matter as described herein. The process 500, or portions thereof, can be performed by or with the aid of one or more computing devices, a computer system, computer-executable instructions, software, hardware, firmware or a combination thereof in various embodiments. For example, portions of process 500 can be performed by a surgical navigation or a robotic system, such as surgical navigation system 200 shown in FIGS. 3-5, robotic system 300 shown in FIG. 6, robotic system 410 shown in FIG. 7, and/or other similar systems.

As shown in process 500, a rotating tool can be positioned 501 so that the tip is adjacent to a work surface where a cut is to be made according to a surgical plan. In embodiments, the rotating tool can be the rotating tool 214 shown in FIGS. 3-5, the rotating tool 314 shown in FIG. 6, or the rotating tool 414 shown in FIG. 7. The rotating tool can be a drill, a bur, or other similar tool or surgical instrument. The work surface can be a bone or other similar work surface.

The rotating tool can be aligned 502 to provide for advancing the rotating tool tip along a predetermined path. In certain embodiments, the rotating tool can be aligned using alignment module 222 shown in FIGS. 3-5 and/or alignment module 316 shown in FIG. 6. In other embodiments, the crosshair interface 228 of FIG. 3 or 322 is FIG. 6 is used.

The rotating tool can be activated 503 to advance the rotating tool tip outside of a cleated sleeve and toward the work surface. In embodiments, the rotating tool can be activated by trigger 224 shown in FIGS. 3-5 or trigger 318 shown in FIG. 6. Alternatively, the rotating tool can be activated by the robotic system after a surgeon waits a predetermined period of time with the tool in a proper alignment for a predetermined trajectory. In another embodiment, the rotating tool is not touching the work surface when it is activated and advances into the work surface when the alignment is correct.

A robotic system can monitor 504 whether the rotating tool is advancing along the predetermined path. In embodiments, the movement of the rotating tool can be monitored with the monitor 226 shown in FIGS. 3-5 or the monitor 320 shown in FIG. 6. Once a depth is reached that is consistent with the surgical plan, the monitor ceases the operation of the rotating tool and can withdraw the rotating tool from the bone or work surface automatically or upon release of a trigger by the surgeon.

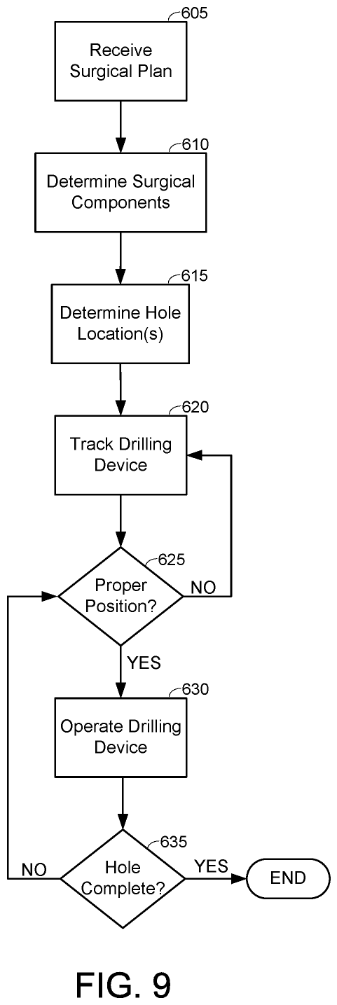

FIG. 9 illustrates a sample process for using a computer-assisted surgical (CAS) system, such as the surgical systems described above, for tracking and controlling the operation of a tool such as the rotating tool described herein. The CAS system can receive 605 a surgical plan including, for example, information related to the surgical procedure such as surgery type (e.g., total knee replacement, partial knee replacement), implant family, implant size, optimal implant location, and other related information. Based upon the surgical plan, the CAS system can determine 610 one or more surgical components that are to be used during the procedure. For example, the one or more surgical components can include cut guides to be mounted to a patient's bone during the surgical procedure to prepare the bone to receive one or more implants. In some examples, the CAS system can determine 610 the surgical components by extracting this information from the surgical plan. In other examples, the CAS system can determine 610 the surgical components based upon a selection of the components by, for example, a surgeon performing the procedure.

The CAS system can also determine 615 hole location information related to holes to be drilled into a patient's bone for mounting, for example, a cut guide to the patient's bone. The CAS system can track 620 the position, orientation, and alignment of a drilling device, such as rotating tool 214 as described above, to determine whether the drilling device is properly positioned to cut the determined holes. If the CAS system determines 625 that the drilling tool is in the proper position, the CAS system can initiate operation 630 of the drilling device. If the CAS system does not determine 625 that the drilling device is in the proper position, the CAS system can continue to track 620 the position of the drilling device.

During operation 630 of the drilling device, the CAS system can monitor the depth of the hole to determine 635 whether the hole is complete. If the hole is complete, the CAS system can stop the operation of the drilling device and either retract the drilling device from the hole or instruct the physician to remove the drilling device (or drilling device tip) from the hole.

If the CAS system does not determine 635 that the hole is complete, the CAS system can determine 625 whether the drilling device is still in the proper position during the drilling. If the CAS system does determine 625 the drilling device is in the proper position, the CAS system can continue operation 630 of the drilling device. If the drilling device is no longer in the proper position, the CAS system can continue to track 620 the position of the drilling device.

It should be noted that the process as shown in FIG. 9 is shown by way of example only and has been simplified for explanatory purposes. For example, the CAS system can continually track the movement and position of the drilling device during operation and can stop operation of the drilling device at any point when the position or alignment of the drilling device is improper. Similarly, rather than merely tracking the drilling device if the drilling device is out of position, the CAS system can adjust the position of the tip of the drilling device as described above to properly position and align the drilling device for drilling the hole(s) in the target bone.

The techniques and processes as described herein can be implemented within a navigation system that can correlate a planned implant location relative to the bone with a database that contains the necessary pin trajectories associated with each cutting block. Such systems can utilize a surgical plan and coordinate transforms to define where the cut block design aligns with the necessary cuts for the implant. Such systems can determine where the pin trajectories lie within in the cut block, which can be used to determine the proper placement of the pilot holes without the need for manual alignment.

The disclosed invention can be used with such a surgical navigation system to precisely align cutting blocks without the need to utilize a jig that is placed with trackers or couplers to eliminate an unnecessary step in conventional robot-assisted implant surgical procedures. As a result, the invention can allow for the easy alignment of cutting jigs or guides. The cutting jigs or guides can be smaller or less intricate because they do not have to interface with manual instrumentation sets. The use of smaller or less intricate cutting jigs or guides reduces costs and allows for the use of a single system for alignment of multiple types of jigs or guides to support multiple surgeries and implant designs.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A system for controlling operation of a surgical tool during a surgical procedure, the system comprising:
a tool assembly comprising:
a rotating tool, and
a sleeve for holding the rotating tool, wherein the sleeve comprises at least one anchoring feature configured to anchor the tool assembly to a target bone prior to actuation of the rotating tool; and
a surgical system comprising:
a navigation system configured to track at least a portion of the rotating tool and determine position information for the rotating tool,
an alignment module configured to receive the position information and determine whether the rotating tool is in a proper position for drilling a hole into the target bone,
a robotic control component configured to actuate and advance the rotating tool if the alignment module determines the rotating tool is in the proper position, and
a monitor configured to determine if the rotating tool is advancing into the target bone along a predetermined path.

2. The system of claim 1, wherein the surgical system is configured to:
determine a surgical plan for the surgical procedure;
determine at least one hole to be drilled into the target bone according to the surgical plan; and
determine the predetermined path for the at least one hole to be drilled based upon the surgical plan.

3. The system of claim 2, wherein the surgical plan defines a knee replacement procedure.

4. The system of claim 1, further comprising a trigger configured to receive an input from a user and facilitate actuation of the rotating tool by the robotic control component.

5. The system of claim 4, wherein the trigger comprises at least one of a foot pedal operably connected to the surgical system, a vocal trigger operably connected to the surgical system, and a pushbutton integrated into the tool assembly.

6. The system of claim 1, wherein the monitor is further configured to:
monitor alignment of the rotating tool during drilling of the hole; and
if the rotating tool is out of alignment, stop the rotating tool.

7. The system of claim 1, wherein the monitor is further configured to:
determine a depth of the rotating tool during drilling of the hole; and
stop the rotating tool when the rotating tool reaches a predetermined depth.

8. The system of claim 1, wherein the rotating tool comprises a tool tip for cutting the target bone during drilling of the hole.

9. The system of claim 8, wherein the tool tip comprises at least one of a drill bit and a cutting bur.

10. The system of claim 1, wherein the sleeve comprises an elongated bore through which the rotating tool can extend from and be retracted into.

11. The system of claim 1, wherein the sleeve is configured to position the rotating tool away from the target bone prior to actuation of the rotating tool.

12. A device for controlling operation of a surgical tool during a surgical procedure, the device comprising:
a processing device operably connected to a computer readable medium configured to store one or more instructions that, when executed, cause the processing device to:
track at least a portion of a rotating tool during the surgical procedure,
determine position information for the rotating tool,
determine, based upon the position information, whether the rotating tool is in a proper position for drilling a hole into a target bone such that at least a portion of the rotating tool is anchored to the target bone prior to actuation of the rotating tool,
actuate and advance the rotating tool if the rotating tool is in the proper position, and
determine if the rotating tool is advancing into the target bone along a predetermined path.

13. The device of claim 12, wherein the one or more instructions comprise additional instructions that, when executed, cause the processing device to:
determine a surgical plan for the surgical procedure;
determine at least one hole to be drilled into the target bone according to the surgical plan; and
determine the predetermined path for the at least one hole to be drilled based upon the surgical plan.

14. The device of claim 13, wherein the surgical plan defines a knee replacement procedure.

15. The device of claim 12, wherein the one or more instructions comprise additional instructions that, when executed, cause the processing device to:
monitor alignment of the rotating tool during drilling of the hole; and
if the rotating tool is out of alignment, stop the rotating tool.

16. The device of claim 12, wherein the one or more instructions comprise additional instructions that, when executed, cause the processing device to:
determine a depth of the rotating tool during drilling of the hole; and
stop the rotating tool when the rotating tool reaches a predetermined depth.

17. A method for controlling operation of a surgical tool during a surgical procedure, the method comprising:
tracking, by a navigation system operably connected to a surgical system, at least a portion of a rotating tool during the surgical procedure;
determining, by the navigation system, position information for the rotating tool;
receiving, by an alignment module operably connected to the navigation system, the position information;
determining, by the alignment module, whether the rotating tool is in a proper position for drilling a hole into a target bone based upon the position information such that at least a portion of the rotating tool is anchored to the target bone prior to actuation of the rotating tool;
actuating and advancing, by a robotic control component operably connected to the alignment module, the rotating tool if the rotating tool is in the proper position; and
determining, by a monitor operably connected to the robotic control component, if the rotating tool is advancing into the target bone along a predetermined path.

18. The method of claim 17, further comprising:
determining, by the surgical system, a surgical plan for the surgical procedure;
determining, by the surgical system, at least one hole to be drilled into the target bone according to the surgical plan; and
determining, by the surgical system, the predetermined path for the at least one hole to be drilled based upon the surgical plan.

19. The method of claim 18, wherein the surgical plan defines a knee replacement procedure.

20. The method of claim 17, further comprising:
monitoring, by the monitor, alignment of the rotating tool during drilling of the hole; and
if the rotating tool is out of alignment, stopping, by the monitor, the rotating tool.

21. The method of claim 17, further comprising:
determining, by the monitor, a depth of the rotating tool during drilling of the hole; and
stopping, by the monitor, the rotating tool when the rotating tool reaches a predetermined depth.

22. A system for controlling alignment of a surgical tool during a surgical procedure, the system comprising:
a navigation system configured to track at least a portion of the surgical tool and determine position information for the surgical tool; and
an alignment module configured to receive the position information and determine whether the surgical tool is in a proper position for drilling a hole into the target bone.

23. The system of claim 22, further comprising:
a robotic control component configured to control movement of the surgical tool; and
a monitor configured to determine whether the surgical tool is moving onto the target bone along a predetermined path.
